(19) United States
(12) Patent Application Publication
(10) Pub. No.: US 2021/0085789 A1
(43) Pub. Date: Mar. 25, 2021

(54) PHARMACEUTICAL COMPOSITION FOR CANCER TREATMENT

(71) Applicant: SEOUL NATIONAL UNIVERSITY R&DB FOUNDATION, Seoul (KR)

(72) Inventor: Dal-Hee MIN, Seoul (KR)

(21) Appl. No.: 16/603,359

(22) PCT Filed: Apr. 9, 2018
(86) PCT No.: PCT/KR2018/004159

§ 371 Date: Oct. 7, 2019

(30) Foreign Application Priority Data
Apr. 7, 2017 (KR) 10-2017-0045431

(51) Int. Cl. A61K 41/00; A61K 9/70; A61K 31/519; A61K 33/38; A61K 33/34; A61K 33/242; A61K 33/243; A61K 33/26; A61K 33/32; A61K 33/24; A61P 35/00

(52) U.S. Cl. CPC A61K 41/0071; A61K 9/7007; A61K 31/519; A61K 33/38; A61K 33/34; A61P 35/00; A61K 33/243; A61K 33/26; A61K 33/32; A61K 33/24; A61K 41/0061; A61K 33/242

(57) ABSTRACT

A composition for photodynamic therapy or diagnosis includes a photosensitizer-metallic nanosheet complex. In a case of using a folic acid-loaded metallic nanosheet, a poorly-soluble photosensitizer may be effectively loaded, thus to increase solubility and the photosensitizer may not be degraded in blood during in vivo administration but may maintain stability, thereby inhibiting imprudent release of the photosensitizer. Further, the nanosheet entered into the cancer cells may be substantially completely degraded by glutathione (GSH) present at a high concentration in the cancer cells. Therefore, it is expected that using the photosensitizer-metallic nanosheet complex may drastically reduce a dose of an anticancer agent while having little side effects and may enable effective treatment, thereby achieving a novel concept in anticancer therapeutic effects.

### CROSS REFERENCE TO RELATED APPLICATIONS AND CLAIM OF PRIORITY

This application claims benefit under 35 U.S.C. 119(e), 120, 121, or 365(c), and is a National Stage entry from International Application No. PCT/KR2018/004159, filed Apr. 9, 2018, which claims priority to the benefit of Korean Patent Application No. 10-2017-0045431 filed in the Korean Intellectual Property Office on Apr. 7, 2017, the entire contents of which are incorporated herein by reference.

### TECHNICAL FIELD

The present invention relates to a pharmaceutical composition for cancer treatment, including a photosensitizer-metallic nanosheet complex.

[symbol]

### BACKGROUND ART

Photodynamic therapy (PDT) uses interaction between a photosensitizer (PS) as a therapeutic agent and an effective light source, thus to have almost no side effects and is little cytotoxic, and therefore, is considered as an attractive, non-invasive therapeutic strategy capable of treating cancer with substantially little or no drug resistance. In PDT, apoptosis and necrosis through a production of reactive oxygen species (ROCs) including singlet oxygen (SO) may induce cell death. In this case, PS may be activated by selective irradiation at an appropriate wavelength with a power of light, and then, transfer electrons or energy to ambient oxygen. Therefore, in order to obtain high therapeutic effects while minimizing damage to healthy tissues, an effective strategy to deliver PS to a target on cancer is needed.

However, although the photodynamic therapy using a photosensitizer (PS) significantly reduces side effects in existing chemotherapy or radiation therapy and maximizes cancer treatment effects, a problem of skin photosensitivity occurring after PDT has been indicated.

For example, Photofirin®, which has been permitted as the photosensitizer by US Food and Drug Administration and used for cancer treatment, is non-specifically accumulated in normal tissues and remains in eyes, skin, and the like for a long period of time after PDT. Therefore, when a patient is exposed to light, the above photosensitizer exhibits light-sensitive skin side effects that kill normal cells of the eyes. In order to avoid such light-sensitive skin side effects, the patient must endure inconvenience of living in environments without light for six (6) weeks or more after PDT procedure. Further, if PS is accumulated in the normal tissues around the tumor tissue, a target-to-background signal ratio is deteriorated, and therefore it is not efficient in fluorescent imaging diagnosis of tumors using the PS.

Further, most of PS derivatives has poor solubility in aqueous solution and low photo-inducible activity, and is inefficient in intracellular penetration. Under these circumferences, modified photosensitizers having hydrophilic properties to reduce non-specific accumulation in normal tissues such as silica, gold, polymer, upconversion and carbon nanoparticle, etc., have currently been developed. In fact, PS with increased hydrophilic properties has advantages in which non-specific accumulation in the normal cells is reduced and the PS is quickly discharged out of the body, thus to significantly shorten photosensitive duration, however, there is a drawback that the PS should be administered with an increased dose in order to deliver a sufficient amount of PS to tumor tissues. Further, it is difficult to penetrate into the cancer cell, hence causing a disadvantage of deteriorating photodynamic therapeutic efficacy. Therefore, there is a need for development of a novel photodynamic therapeutic agent that increases accumulation rate specific to cancer cells and does not cause side effects while achieving excellent therapeutic effects.

Recently, new two-dimensional materials having unique physical and chemical properties have attention in fields of bio-imaging, biosensors, drug/gene delivery and regeneration medicine. In particular, 2D materials with a sharp shape similar to a blade showed higher accumulation in cytoplasm than materials having less sharp shape. Moreover, in order to achieve effective intracellular delivery of bio-functional molecules such as a drug, a transporter should be capable of long term storage in a cell having high absorption rate and low excretion rate of a substance. Therefore, such a 2D material is considered as one of alternatives to provide a solution for clinical tasks that could not be solved using a material having a smooth surface such as a spherical particle.

Accordingly, in order to overcome limitations of conventional photodynamic therapy, the present inventors have studied to develop a novel photodynamic therapeutic agent that has high stability and is readily degradable under specific environments in vivo, and have found a functional nanosheet that may more easily penetrate into cells than existing nanoparticles and may also selectively recognize cancer cells only, and the present invention has been completed on the basis of the above finding.

[symbol]

### SUMMARY

It is an object of the present invention to provide a pharmaceutical composition for cancer treatment, including a photosensitizer-metallic nanosheet complex.

[symbol]

1. A pharmaceutical composition for cancer treatment, including: a metallic nanosheet degraded in a tumor tissue, wherein a folic acid to be bound to a folate receptor on a surface of a tumor cell and a photosensitizer excited by light irradiation at a predetermined wavelength are distributed on a surface of the metallic nanosheet.

2. The pharmaceutical composition of the above 1,

wherein the metallic nanosheet contains gold, silver, copper, platinum, palladium, nickel, iron, manganese or oxides thereof.

3. The pharmaceutical composition of the above 1,

wherein the metallic nanosheet contains manganese dioxide (MnO₂).

4. The pharmaceutical composition of the above 1,

wherein the metallic nanosheet is introduced into a cancer cell by folate (FA) receptor-mediated endocytosis.

5. The pharmaceutical composition of the above 1,

wherein the photosensitizer is selected from the group consisting of phthalocyanine compounds, porphyrin compounds, chlorine compounds, bacteriochlorine compounds, naphthalocyanine compounds and 5-aminolevuline ester compounds.

6. The pharmaceutical composition of the above 1,

wherein the photosensitizer is zinc-phthalocyanine (ZnPc).

7. The pharmaceutical composition of the above 1,

wherein the predetermined wavelength ranges from 600 nm to 800 nm.

8. The pharmaceutical composition of the above 1,

wherein the excited photosensitizer generates singlet oxygen or free radicals.

9. The pharmaceutical composition of the above 1,

wherein the photosensitizer is contained in an amount of 5 to 20% by weight based on a total weight of a folic acid-metallic nanosheet-photosensitizer complex.

10. The pharmaceutical composition of the above 1,

wherein the metallic nanosheet contains manganese dioxide (MnO₂), and the photosensitizer is zinc-phthalocyanine (ZnPc).

11. The pharmaceutical composition of the above 10,

wherein the zinc-phthalocyanine is bound to the metallic nanosheet through a Mn—N coordinate bond.

12. The pharmaceutical composition of the above 10, wherein the photosensitizer is bound to the metallic nanosheet by mixing particle size-reduced powders with a FA—MnO₂ solution and agitating the mixture.

13. The pharmaceutical composition of any one of the above 1 to 12,

wherein the cancer is any one or more selected from the group consisting of skin cancer, oral cancer, gastric cancer, ovarian cancer, breast cancer, osteosarcoma, colon cancer, esophageal cancer, duodenal cancer, renal cancer, lung cancer, pancreatic cancer, cervical cancer and prostate cancer.

14. A method for manufacturing a pharmaceutical composition for cancer treatment, including:

distributing folic acid on a surface of a metallic nanosheet; and distributing a photosensitizer on the surface of the metallic nanosheet having the folic acid distributed thereon.

15. The method of the above 14, wherein the metallic nanosheet contains MnO₂, and the photosensitizer is zinc-phthalocyanine.

16. The method of the above 15, further including reducing a particle size of the photosensitizer powder before distribution of the photosensitizer.

[symbol]

The present invention relates to a composition for photodynamic therapy or diagnosis, including a photosensitizer-metallic nanosheet complex. In a case of using the folic acid-loaded metallic nanosheet of the present invention, a poorly-soluble (commonly referred to as ‘insoluble’) photosensitizer may be effectively loaded, thus to increase solubility and the photosensitizer may not be degraded in blood during in vivo administration but may maintain stability, thereby inhibiting imprudent release of the photosensitizer. Further, the nanosheet entered into the cancer cells may be substantially completely degraded by glutathione (GSH) present at a high concentration in the cancer cells. Therefore, it is expected that using the photosensitizer-metallic nanosheet complex may drastically reduce a dose of an anticancer agent while having little side effects and may enable effective treatment, thereby achieving a novel concept in anticancer therapeutic effects.

The pharmaceutical composition of the present invention may exhibit high anticancer effects even by administering 10% the dose of the conventional photosensitizer-containing anticancer agent.

[symbol]

### BRIEF DESCRIPTION OF THE DRAWINGS

FIG. 1 is a schematic diagram illustrating FA—MnO₂-mediated ZnPc delivery of the present invention and a process for targeted photodynamic therapy (PDT) according to the above delivery.

FIG. 2 is views illustrating characteristics of FA-MnO₂ of the present invention, wherein a) is AFM topographical image of FA-MnO₂; b) is TEM image of FA-MnO₂; c) is constitutional compositions of FA- MnO₂ and MnO₂ by XPS assay; d) is UV-Vis-NIR absorption spectra of FA-MnO₂ and MnO₂; and e) is FT-IR spectrum.

FIG. 3 is views illustrating characteristics of FA-MnO₂/ZnPc complex of the present invention, wherein a) is UV-Vis-NIR absorption spectra of ZnPc and FA-MnO₂/ZnPc; and b) is time-dependent recovery of fluorescent intensity of ZnPc from FA-MnO₂/ZnPc at an emission wavelength of 670 nm under various conditions.

FIGS. 4A to 4C are views illustrating intracellular uptake of FA-MnO₂/ZnPc complex of the present invention, wherein FIG. 4A is bright fields and fluorescent images of HeLa cells treated with MnO₂/ZnPc, FA-MnO₂ or FA-MnO₂/ZnPc; FIG. 4B is a cell population histogram obtained by flow cytometric analysis; and FIG. 4C is a bar graph of relative fluorescence intensity corresponding to the above histogram.

FIGS. 5A to 5C are views illustrating in vitro photodynamic effects of FA-MnO₂/ZnPc complexes of the present invention, wherein FIG. 5A is bright fields and fluorescent images of HeLa cells treated with MnO₂ derivatives after laser irradiation; FIG. 5B is time-dependent fluorescence intensity of SOSG at 530 nm (λex=504); and FIG. 5C is relative survival rates of HeLa cells after administering a small amount of FA-MnO₂/ZnPc or ZnPc at different concentrations.

FIGS. 6A to 6C are views illustrating in vivo targeting and photodynamic anti-cancer effects of FA-MnO₂/ZnPc complex of the present invention, wherein FIG. 6A is bright fields and fluorescent images 12 hours after intravenous injection of FA-MnO₂/ZnPc, MnO₂/ZnPc or PBS to tumor-xenograft mice; FIG. 6B is time-dependent relative tumor volumes in the tumor-xenograft mice treated with PBS, FA-MnO₂, MnO₂/ZnPc or FA-MnO₂/ZnPc during light irradiation; and FIG. 6C shows results of H&E staining tumor sections, 2 weeks after injection of PBS, FA-MnO₂, MnO₂/ZnPc or FA-MnO₂/ZnPc.

FIG. 7 is views illustrating AFM topographical images of a) FA-MnO₂/ZnPc and b) MnO₂/ZnPc.

FIG. 8 is views illustrating bright fields and fluorescent images of MDA-MB-231 cells (FR-overexpressing cells) and A-549 cells (FR-deficient cells).

FIG. 9 is views illustrating ex vivo anticancer effects of FA-MnO₂/ZnPc complex of the present invention, wherein bright fields and fluorescent images of a) tumor and b) main organs, 12 hours after intravenous injection of FA-MnO₂/ZnPc, MnO₂/ZnPc or PBS to the tumor-xenograft mice, are illustrated.

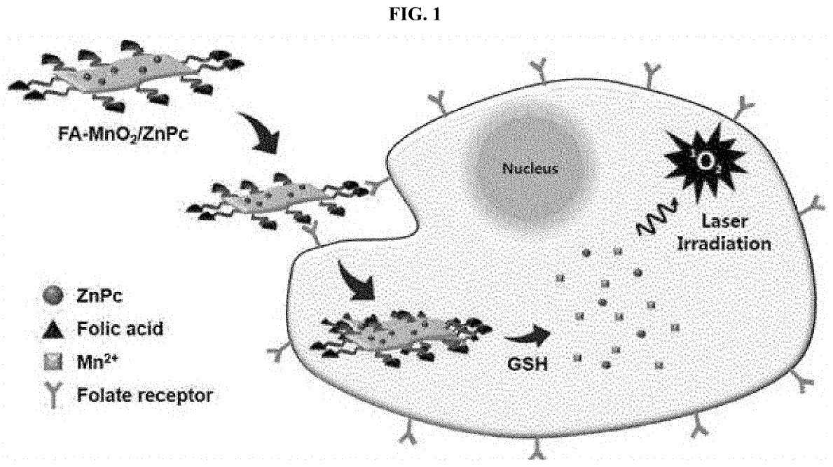

[symbol]

### DETAILED DESCRIPTION

In preferred embodiments, the present inventors have practically verified that the folic acid-loaded photosensitizer-metallic nanosheet complex (FA-MnO₂/ZnPc) have intracellular penetration ability, tumor targeting ability and are easily degradable in specific environments in vivo and, and the present invention has been completed on the basis of the verified facts.

[symbol]

Hereinafter the present invention will be described in detail.

[symbol]

The present invention provides a pharmaceutical composition for cancer treatment, including a metallic nanosheet, wherein folic acid to be bound to a folate receptor on a surface of a cancer cell and a photosensitizer excited by light irradiation at a predetermined wavelength are distributed on a surface of the nanosheet, and the metallic nanosheet is degraded in tumor tissues.

The metallic nanosheet according to the present invention may contain gold, silver, copper, platinum, palladium, nickel, iron, manganese or oxides thereof, and preferably, manganese dioxide. In particular, the manganese dioxide has excellent solubility in an aqueous solution, strong interaction with small molecules and biopolymers, and high biocompatibility based on degradation and excretion without unexpected accumulation in vivo. Further, the inventive nanosheet has advantages of: superior drug loading efficiency based on very wide surface area as compared to a mass thereof; and excellent extinction capability of fluorescent signals from the loaded substance by absorbing light in a wide wavelength range. Therefore, the nanosheet may be used as an effective delivery system of the photosensitizer. In addition, when using along with a zinc-phthalocyanine photosensitizer, the nanosheet may be strongly bound through coordinate bond between N in phthalocyanine and Mn in manganese dioxide.

Folic acid and the photosensitizer may be distributed on the surface of the metallic nanosheet.

Folic acid may be, for example, loaded on the metallic nanosheet or coupled thereto through electrostatic interaction between the metallic nanosheet and folic acid, coordinate bond, covalent bond, etc., thus being distributed on the surface of the metallic nanosheet, but it is not limited thereto. Since cancer cells have a large amount of folate receptor on the surface thereof, the metallic nanosheet having folic acid distributed on the surface thereof may be combined with the surface of the cancer cell, which in turn may be introduced (referred to as ‘transfected’) into the cancer cell by endocytosis.

The photosensitizer may be loaded by a variety of intermolecular interactions, for example, hydrophobic interaction, pi-pi stacking interaction, electrostatic interaction, hydrogen bond, coordinate bond, covalent bond, etc. between the metallic nanosheet and the photosensitizer.

The photosensitizer according to the present invention may include, for example, phthalocyanine compounds, porphyrin compounds, chlorine compounds, bacteriochlorine compounds, naphthalocyanine compounds or 5-aminolevuline ester compounds, and particularly, zinc-phthalocyanine (ZnPc). Herein, the phthalocyanine photosensitizer may exist in a coordination bond form with most of metal ions. Especially, since zinc is a significant element necessary for major hormones, enzymes and immune function in the human body, zinc-phthalocyanine may be easily synthesized and have an economical merit. Further, zinc-phthalocyanine has a high absorption coefficient and may be available for efficient photodynamic therapy even with a small amount thereof.

Zinc-phthalocyanine is an insoluble material and very limited in using as a drug. However, according to the present invention, if the nanosheet contains manganese dioxide, zinc-phthalocyanine may also be used to form a Mn—N coordinate bond and thus can be loaded, thereby remarkably increasing drug loading efficiency.

Particular examples of using zinc-phthalocyanine as a photosensitizer are as follows. Zinc-phthalocyanine in a powder form is commonly available in the market (e.g., a produce from Sigma-Aldrich Co.). In the present invention, zinc-phthalocyanine having a reduced particle diameter may be used. For example, the powder of zinc-phthalocyanine may have a particle diameter of 10 μm or less, 5 μm or less, 2.5 μm or less, etc. In such a case, when using the zinc-phthalocyanine along with a manganese dioxide nanosheet, zinc-phthalocyanine molecules may form a larger amount of coordinate bonds, thereby remarkably increasing the loading efficiency of the nanosheet. The zinc-phthalocyanine photosensitizer may be prepared by decreasing the particle size of zinc-phthalocyanine powder (reduction of particle size), for example, by sieving the powders, filtering the powders through a fine filter, re-grinding the powders, or dispersing the powders in a solution and then evaporating a dispersion, but it is not limited thereto.

The photosensitizer according to the present invention does not show toxicity in a ground state. However, when absorbing light at a specific wavelength, the photosensitizer is excited to a singlet state. Although a part of the photosensitizer in the singlet state returns to the ground state while radiating energy in a fluorescent light form, the greater part thereof may be transferred to a triplet state through intersystem crossing. The photosensitizer in such a singlet state or triplet state as described above may react with a surrounding substrate or oxygen around the photosensitizer to generate reactive oxygen species, for example, singlet oxygen, oxygen radicals, super oxide or peroxide. The generated reactive oxygen species may enable apoptosis or necrosis of surrounding tumor cells.

In consideration of optical tissue penetration potential and efficiency of generating the reactive oxygen species, the photosensitizer may be excited by light irradiation at a wavelength range of 450 to 950 nm, and preferably, 600 to 800 nm, thereby generating singlet oxygen or free radicals.

The metallic nanosheet of the present invention may be included in the above composition in a form of a photosensitizer-metallic nanosheet complex, in which folic acid and the photosensitizer are distributed on a surface thereof. During circulation in blood following administration of the composition to the body, the photosensitizer is not dissociated from the metallic nanosheet, but may retain the complex form.

The photosensitizer in the complex may be contained in a content of 5 to 30% by weight (‘wt. %’), for example, 5 to 30 wt. %, 5 to 20 wt. %, 5 to 15 wt. %, 5 to 10 wt. %, etc., based on a total weight of the complex, but it is not limited thereto.

Further, the complex is larger than the photosensitizer itself and has a difficulty in penetrating into normal tissues having a dense vessel wall, thereby maintaining stability. However, the complex can be transfected into tumor cells by FA receptor-mediated endocytosis excessively expressed in the tumor, such that the complex may be very easily accumulated in the tumor cells. As a result, the complex may be specifically accumulated in the tumor tissue.

With regard to the complex specifically accumulated in the tumor tissue, the photosensitizer is gradually separated and thus dissociated from the metallic nanosheet over time. Thereafter, when the photosensitizer is exposed to light having a wavelength at which the photosensitizer can be excited, the photosensitizer may be excited in a singlet state. The photosensitizer in the singlet state may react with the surrounding substrate or oxygen to generate reactive oxygen species. The generated reactive oxygen species may induce apoptosis or necrosis of surrounding tumor cells. As such, as the complex specifically accumulated in the tumor tissues is gradually dissociated, the tumor tissue may be efficiently destroyed during photodynamic therapy and, at this time, the metallic nanosheet may be readily degraded by a reducing agent such as glutathione present in the tumor cells, with or without loading of photosensitizer. Further, due to degradation of the metallic nanosheet, the loaded drug (or photosensitizer) may be released. Furthermore, tumor diagnosis may be performed through selective fluorescence generation in the tumor tissue.

Cancer referred in the present disclosure may be all cancers resulting from tumor cells expressing folate receptors on the surface thereof. For example, the cancer may include skin cancer, oral cancer, gastric cancer, ovarian cancer, breast cancer, osteosarcoma, colon cancer, esophageal cancer, duodenal cancer, renal cancer, lung cancer, pancreatic cancer, cervical cancer and prostate cancer, but it is not limited thereto.

The pharmaceutical composition of the present invention may be prepared and/or administered to a mammal using a pharmaceutically suitable and physiologically acceptable adjuvant other than the above active ingredients. The adjuvant used herein may include, for example, excipients, disintegrants, sweeteners, binders, coating agents, swelling agents, lubricants, glidants or flavoring agents.

Further, for administration purpose, the pharmaceutical composition of the present invention may further include at least one of pharmaceutically acceptable carriers other than the above active ingredients in pharmaceutically effective amounts, thereby being preferably prepared in various pharmaceutical formulations.

Herein, the term “pharmaceutically effective amount” as used herein refers to an amount sufficient to treat a disease at a reasonable benefit/risk ratio, which is applicable to any medical treatment, and an effective dose level may be determined according to diverse factors such as types of diseases of a patient, severity, activity of a drug, drug sensitivity, administration time, administration route, discharge rate, treatment duration, coexistence of other drugs, and other factors well known in medical fields. The pharmaceutical composition according to the present invention may be administered as a single therapeutic agent or in combination with other therapeutic agents. Further, the inventive composition may be administered sequentially or simultaneously with conventional therapeutic agents, and may be administered in a single dose or multiple doses. It is important to administer the composition in a minimum amount capable of accommodating maximum effects without side effects, which may be readily determined by those skilled in the art in consideration of all the factors described above.

More particularly, an effective amount of the pharmaceutical composition of the present invention may vary on the basis of age, gender, conditions or body weight of a patient, absorption of active ingredients in vivo, rate of inactivity, excretion rate, types of diseases, drugs used along therewith, etc. Generally, the composition may be administrated in an amount of 0.001 to 150 mg, preferably, 0.01 to 100 mg per 1 kg of body weight every day or every other day, or in 1 to 3 times a day. However, the dose may be increased or decreased depending upon the administration route, severity of obesity, gender, body weight, age, etc. of the patient, and is not intended to limit the scope of the present invention in any manner.

Further, the term “pharmaceutically acceptable” as used herein typically refers to a composition that does not cause allergic reaction or reactions similar thereto such as gastrointestinal disorder, dizziness, etc. when administering to a human. The carriers, excipients and diluents described above may include, for example, lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinylpyrrolidone, water, methyl hydroxybenzoate, propyl hydroxybenzoate, talc, magnesium stearate and mineral oil. Further, fillers, anti-coagulants, lubricants, wetting agents, flavoring agents, emulsifying agents and/or preservatives may also be included.

In addition, the composition of the present invention may be formulated by any method well known in the art, in order to accommodate quick, sustained or delayed release of active ingredient after administered to a subject including humans in need of treatment of cancer. The formulation may include, for example, powder, granules, tablets, emulsion, syrup, aerosol, soft or hard gelatin capsules, sterile injection type solutions, sterile powders, etc.

[symbol]

The present invention also provides use of the composition described above as a cancer therapeutic agent.

Further, the present invention provides a method for treatment of a cancer, comprising administration of the above composition in a therapeutically effect amount to a mammal.

The term “mammal” as used herein refers to a mammal as a subject of treatment, observation or experiment, and preferably means a human being. Further, the term “therapeutically effective amount” as used herein refers to an amount of the active ingredient or the pharmaceutical composition to induce a biological or medical response in a tissue system, animal or human, which is determined by a researcher, veterinarian, medical doctor or other clinical doctors, and may include an amount of inducing alleviation of symptoms in disease or disorder to be treated.

[symbol]

Further, the present invention provides a method of manufacturing a pharmaceutical composition for cancer treatment.

The method of manufacturing a pharmaceutical composition for cancer treatment according to the present invention may include: distributing folic acid on a surface of a metallic nanosheet; and distributing a photosensitizer on the surface of the metallic nanosheet having the folic acid distributed thereon.

Distributing folic acid on the surface of the metallic nanosheet may be performed by, for example, mixing folic acid in a solvent in which the metallic nanosheet is dispersed or dissolved, followed by agitating the same.

Metal in the metallic nanosheet may be any one of the above-exemplified metals, and preferably, MnO₂.

The solvent used herein may be, for example, water, but it is not limited thereto.

Agitation may be conducted by, for example, ultra-sonication, but it is not limited thereto.

The agitation may be conducted, for example, at a temperature of 0 to 30° C., but it is not limited thereto.

If necessary, the inventive method may further include distributing folic acid on the surface of the metallic nanosheet and then filtering the same.

The filtration may be conducted by dialysis through a membrane of about 1 kDa to 50 kDa, but it is not limited thereto. Particularly, dialysis may be performed using a membrane of 2 kDa to 20 kDa, and more particularly, a membrane of 5 kDa to 15 kDa, but it is not limited thereto.

Following this, the photosensitizer may be distributed on the surface of the metallic nanosheet having the folic acid distributed thereon. Accordingly, the folic acid may be distributed on a part of the surface of the metallic nanosheet, while the remaining part thereof is distributed with the photosensitizer.

Distribution of the photosensitizer on the surface of the metallic nanosheet having the folic acid distributed thereon, may be performed by, for example, mixing the photosensitizer with a solvent in which the metallic nanosheet having the folic acid distributed thereon is dispersed or dissolved, and then agitating the mixture.

The photosensitizer used herein may include those exemplified above, preferably, zinc-phthalocyanine.

For specific example, zinc-phthalocyanine may be in a powder form, preferably, that was subjected to reduction of a particle size. Accordingly, the method of the present invention may further include reducing a particle size of zinc-phthalocyanine. In this case, when using the zinc-phthalocyanine along with MnO₂ metallic nanosheet, binding between zinc-phthalocyanine molecules and the metallic nanosheet may be increased, thereby further improving loading efficiency.

Particle size reduction is a process of decreasing a particle diameter of the zinc-phthalocyanine powder, and any known method used for reduction of the particle diameter of the zinc-phthalocyanine powder may be employed without limitation thereof. For example, the particle size reduction may be performed in such a manner as sieving, filtration using a fine filter, re-grinding, evaporation of a dispersion containing the powders, etc., but it is not limited thereto.

The reduction of particle size may be performed, for example, so as to attain a powder particle diameter of 10 μm or less, 5 μm or less, or 2.5 μm or less, but it is not limited thereto.

The solvent may be, for example, water, but it is not limited thereto.

Agitation may by conducted, for example, by ultra-sonication, but it is not limited thereto.

The agitation may be conducted, for example, at 0 to 30° C., but it is not limited thereto.

If necessary, the method of the present invention may further include distributing the photosensitizer on the surface of the metallic nanosheet having the folic acid distributed thereon, and then filtering the same.

The filtration may be conducted, for example, using a filter of 0.01 to 1 μm, and more particularly, a filter of 0.1 to 0.5 μm, but it is not limited thereto.

[symbol]

Hereinafter, preferred embodiments of the present invention will be described by means of following examples, in order to concretely describe the invention. However, these examples are provided only for more easily understanding the present invention and it is duly appreciated that the present invention would not be particularly limited to the examples.

[symbol]

### EXAMPLE

The present examples were implemented to determine whether a folic acid (FA)-bound MnO₂ (FA-MnO₂) nanosheet can be used as a carrier of photosensitizer (PS) for photodynamic therapy (PDT) and a bio-imaging system, and to verify superiority of the FA-MnO₂ nanosheet by analyzing effects of the photodynamic therapy through cell experiments, in order to manufacture an effective composition for cancer treatment using the above nanosheet.

[symbol]

### 1. TEST PREPARATION AND TEST PROCEDURE

### 1-1. Preparation of Sample

FA (Folic acid) and ZnPc (zinc phthalocyanine) were purchased from Sigma-Aldrich (USA) and a CCK-8 analysis kit was purchased from Dojino Molecular Technology (USA). A live/dead viability/cytotoxicity kit for mammalian cells (Calcein AM and ethidium homodimer-1) and a singlet oxygen sensor green (SOSG) were purchased from ThermoFisher Scientific (USA). Further, PBS (Phosphate buffered saline, 10x), DMEM (Dulbecco's modified eagle's medium), RPMI (Rosewell park memorial institute) 1640, FBS (fetal bovine serum) and P/S (penicillin and streptomycin, 100x) were purchased from WELGENE (Korea).

[symbol]

### 1-2. Characterization

UV-vis spectrum was obtained with UV-2550 (Shimadzu, Japan). In order to ascertain chemical modification of functional groups, FT-IR spectrum was characterized through Nicolet iSTM 10 FT-IR spectrometer (Thermo Fisher Scientific, USA) using KBr pellets. A morphological image and a thickness were determined by using an atomic force microscope (AFM), an aluminum probe having a reflex-coated rear surface at a thickness of 30 nm (a non-contact cantilever), XE-100 (Park System, Korea), and a transmission electron microscope (TEM, LIBRA 123, Carl Zeiss). Further, XPS (AXIS-HSi, Shimadzu, Japan) was conducted to analyze the elemental composition of a material, while a hydrodynamic radius and zeta potential were measured by zetasizer NS90 (Malvern, UK).

[symbol]

### 1-3. Preparation of FA-MnO₂ nanosheet and FA-MnO₂/ZnPc complex

The prepared MnO₂ (1 mg ml⁻¹) was mixed with FA (50 mM) in an aqueous solution and the mixture was subjected to ultra-sonication in an ice bath for 2 hours, followed by agitation at room temperature for 1 hour and dialysis through 10 kDa membrane overnight. In order to prepare FA-MnO₂ with ZnPc, ZnPc added thereto was mixed with FA-MnO₂ solution and the mixture was subjected to ultra-sonication in the ice bath for 1 hour, followed by filtration using 0.2 μm syringe filter made of polyvinyl difluoride (PVDF) (Merck Millipore, USA). Herein, a loading capacity of ZnPc in FA-MnO₂-ZnPc complex, which was calculated by using UV-vis spectrum spectroscopy and ZnPc standard absorbance curve, was not more than 8 wt. %. ZnPc was used after filtration using a sieve of 50 mesh (about 2.5 μm) before the above mixing step.

Further, according to the fluorescence emission spectrum of ZnPc (0.4 μM) obtained at an excitation wavelength of 650 nm using Synergy MX fluorescence measurement device (BioTek, Inc.) in the presence or absence of FAMnO₂ (1.0 mg ml⁻¹), loading of ZnPc to FA-MnO₂ was assessed. ZnPc release from FA-MnO₂ was determined from the recovered florescence of ZnPc in reducing conditions. GSH and DTT (10 mM) were added to FAMnO₂/ZnPc solution, followed by measuring fluorescence intensity at 670 nm every 30 minutes. Time-dependent fluorescent spectra were plotted in proportion to initial fluorescence of ZnPc, and BSA and FBS added samples were used as control groups.

[symbol]

### 1-4. Cell Culture

A human cervical cancer cell line HeLa and a breast cancer cell line MDA-MB-231 were cultured in DMEM containing 10% FBS and 1% P/S at 37° C. under 5% CO₂ condition. Further, a human alveolar basal epithelial cell line A-549 was cultured in RPMI 1640 containing the same components under the same conditions as described above.

[symbol]

### 1-5. Assessment of Cell Viability

In order to determine viability of cells treated with FA-MnO₂, HeLa cells (1×10⁴ cells/well) were prepared in three (3) 96-well plates for 24 hours, and cultured with FA-MnO₂ at different concentrations as well as a complete medium. After incubation for 12 hours, the cells were gently washed with 1×PBS and CCK-8 analysis solution as well as a serum-free medium were added thereto for one hour, followed by measuring absorbance at wavelengths of 450 nm and 670 nm using a micro-plate reader (Molecular Devices, Inc., USA).

[symbol]

### 1-6. Cell Imaging and Flow Cytometry

HeLa cells (1.2×10⁵ cells/well) were incubated on 4-well glass plate for 24 hours, and MnO₂ derivatives were added to each well in the serum-free medium for 12 hours. In order to identify effects of FR-mediated cell uptake, the cells were treated with free-FA (10 mM) for 2 hours before FA-MnO₂/ZnPc (50 μg ml⁻¹) treatment. After incubation for 12 hours, the cells were gently washed with 1×PBS and the medium was substituted with a fresh medium containing serum. MDA-MB-231 cells and A-549 cells were prepared in the same procedure as described above except for free-FA treatment.

According to the protocol of the manufacturer, nuclei were stained by Hoechst 33342 staining kit. Then, bright fields and fluorescence images were obtained by DeltaVision Elite Microscopy and In Cell Analyzer 2000 (GE Healthcare, USA), and the obtained images were used in a software provided for pseudo-coloring, fluorescence intensity and background removal processes.

In order to quantitatively analyze a mean fluorescence intensity of ZnPc, the HeLa cells treated with MnO₂ derivatives were collected for 3 minutes after trypsin-EDTA treatment and 10% FBS was added to the collected cells, followed by centrifugation (1200 rpm, 3 minutes). Finally, the cells were washed with 1×PBS, and fluorescence of the cells was determined by a flow cytometric fluorescence measurement device (FACS Canto, Beckton Dickinson Bioscience, USA). The collected flow cytometric data were analyzed by FlowJo software.

[symbol]

### 1.7 (In Vitro) Photodynamic Therapy

In order to identify therapeutic effects of in vitro PDT (photodynamic therapy), pre-treated HeLa cells (1.2×10⁵ cells/well) were treated with FA-MnO₂/ZnPc (50 μg ml⁻¹) in a serum-free medium in 12-well plate for 24 hours. After replacing the medium with a serum-containing medium, the cells was irradiated with 660 nm fiber-coupled laser (LaserLab, Korea, 30 mW cm⁻²) for 10 minutes. Additionally, after incubation for 12 hours, each well was treated with Live/Dead analysis reagent based on a protocol of the manufacturer. A bright field and a fluorescence image of the cells were obtained by means of an inverse fluorescence microscope having 4X objective lens (Olympus, Japan).

Next, in order to determine quantitative cell viability associated with photodynamic effects of FA-MnO₂/ZnPc, HeLa cells (1×10⁴ cells/well) were seeded on a 96-well plate, incubated for 24 hours, and then, treated with FA-MnO₂/ZnPc and ZnPc at various concentrations. After the incubation for 12 hours, each well was irradiated with 660 nm LED (Mikwang Electronics, Korea) at 30 mW cm⁻² for 10 minutes. After additional incubation in a serum-containing medium for 12 hours, analysis of CCK-8 cell vitality was assessed in the same manner as described above.

All experiments have been conducted in triplicate.

[symbol]

### 1-8. Singlet Oxygen Detection

SOSG reagent is highly selective for SO and emits strong green fluorescence in the presence of SO at 530 nm (λₑₓ=504 nm). After adding SOSG (5.0 μM) dissolved in 2% methanol solution to ZnPc (3.2 μM) and FA-MnO₂/ZnPc (1.0 mg ml⁻¹), GSH (10 mM) was further added as necessary. A final volume of each well was 100 ml. SO generation was induced by irradiation using 660 nm LED (light emitting diode) (30 mW cm⁻²). After irradiation, green fluorescence emission from a sample was observed at a wavelength of 530 nm for 55 minutes by using a fluorescence measurement device.

[symbol]

### 1-9. In Vivo Imaging-Based Biological Classification and Anticancer Effects of PDT

All animal experiments were performed in compliance with protocols approved by the Institutional Animal Care and Use Committee (IACUC) of Seoul National University. Balb/c male nude mice (5 weeks old) were purchased from ORIENT BIO (Korea). A mouse having tumor was prepared by subcutaneous injection of a suspension including 100 μl of 1×PBS (n=4) sterile solution as well as HeLa cells (6×10⁶ cells). When the tumor size reaches about 50 mm³, MnO₂/ZnPc and FA-MnO₂/ZnPc in 1×PBS solution (0.5 mg ZnPc/kg) were injected into a tail vein of the mouse having the tumor. As a control group, a group of mice were treated with the same volume of 1×PBS solution. Bright fields and fluorescent images were obtained using Optix MX3 (ART, USA) 12 hours after injection. In order to identify bio-distribution of the injected ZnPc, 12 hours after the injection, major organs were collected in a Petri-dish. All gained images were used for pseudo-coloring with the fluorescence intensity.

For determination of anticancer effects, each of ZnPc containing MnO₂ and FA-MnO₂ (0.5 mg ZnPc/kg), respectively, was prepared in a final volume of 100 μl, and 1×PBS was used as a control group. When a tumor volume in HeLa-implanted xenograft mouse reaches about 50 mm³ (n=4), the suspension was injected. 12 hours after the injection, 660 nm fiber-coupled laser (0.2 W cm⁻², 10 minutes) was used for irradiation, followed by monitoring each group on the tumor volume and a change in body weight over 2 weeks. The tumor volume was estimated according to an equation of length×(width)²×½ wherein the length and the width are a longest diameter and a shortest diameter of the tumor, respectively. A relative tumor volume was calculated, as compared to an initial volume.

[symbol]

### 1-10. Histological Evaluation

At day 14 after intravenous injection, PDT was conducted and the mouse was sacrificed to perform histological evaluation. Samples of the heart, liver, spleen, lungs, kidneys and the tumor were taken, put into 4% PFA solution, embedded in a sucrose-penetrated optimal cutting temperature (OCT) compound and cut into sections, followed by H&E staining (BBC Biochemical, Mt Vernon, Wash., USA). The stained sections were observed by a BX71 microscope with a 10X objective lens (Olympus, Japan).

[symbol]

### 1-11. Statistical Analysis

All data refer to compensated values ±SD of at least four independent experiments, and significant differences were determined by Student's t-test performed using GraphPad software.

[symbol]

### Example 1

### 1. Preparation of FA-MnO₂ Nanosheet

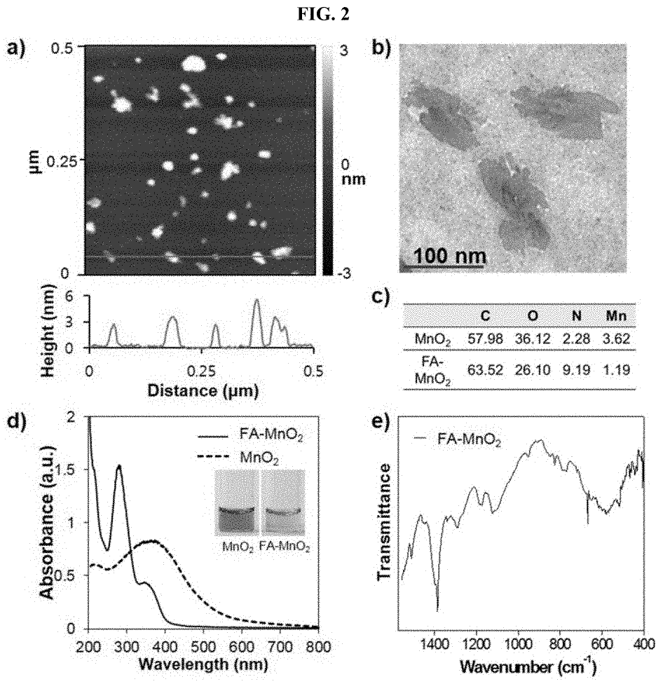

Most of all, according to the method in the above section 1-3, MnO₂ was synthesized by partially modifying the previously known procedure, and then, the prepared MnO₂ nanosheet was characterized according to XPS, AFM, TEM and FTIR assays. Thereafter, FA was bonded to the MnO₂ nanosheet. Characteristics of such fabricated FA-MnO₂ nanosheet are shown in FIG. 2.

First, FA-MnO₂ nanosheet having an average height of about 3 to 10 nm was identified through morphological images having a specific line profile obtained from the AFM and TEM images (see a) and b) of FIG. 2). Further, after binding FA to MnO₂, it was demonstrated that elemental compositions of carbon and nitrogen were significantly increased by 63.5 and 9.19%, respectively. Further, it was identified that sharp UV-absorption peaks corresponding to FA are exhibited at 282 nm along with characteristic peaks at 370 nm due to MnO₂ (see c) and d) of FIG. 2). Finally, FT-IR shows unique peaks of 515 cm⁻¹ (Mn—O) and 479 cm⁻¹ (Mn—N), as well as FA-based additional transmission peaks of 963 cm⁻¹ O—-H bond) and 900 to 750 cm⁻¹ (C—H aromatics) (see e) of FIG. 2). In addition, it was found that a zeta potential value was changed from −19.43±0.63 mV to −32.1±0.1 mV after binding FA to Mn0₂.

As a result of the above descriptions, it could be recognized that FA-Mn0₂ nanosheet was successfully produced.

[symbol]

### 2. Preparation of FA-MnO2/ZnPc Complex

A mixed solution of FA-MnO₂ and ZnPc (photosensitizer, corresponding to PS) prepared in Example was subjected to simple ultra-sonication, thereby producing a ZnPc and FA-MnO₂ complex (FA-MnO₂/ZnPc).

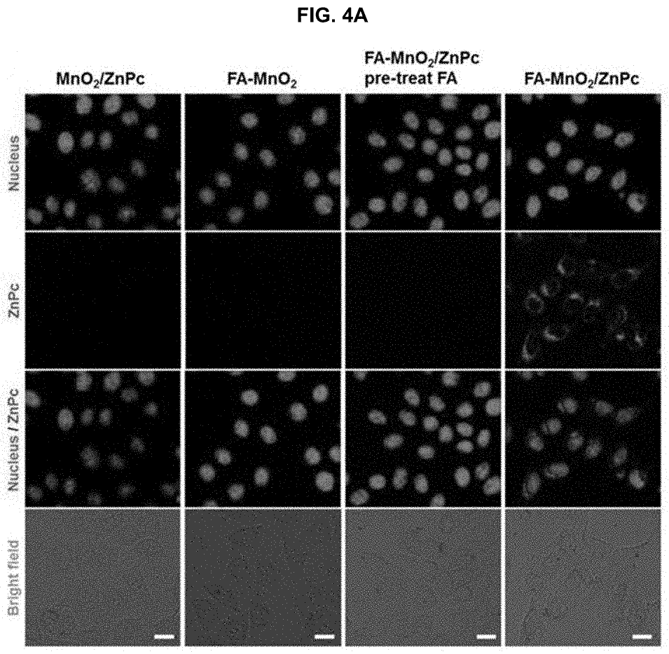

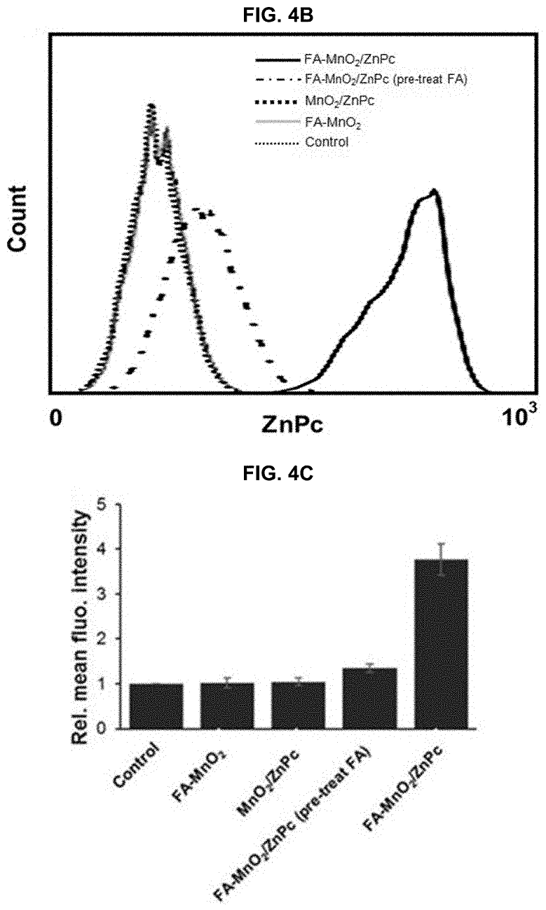

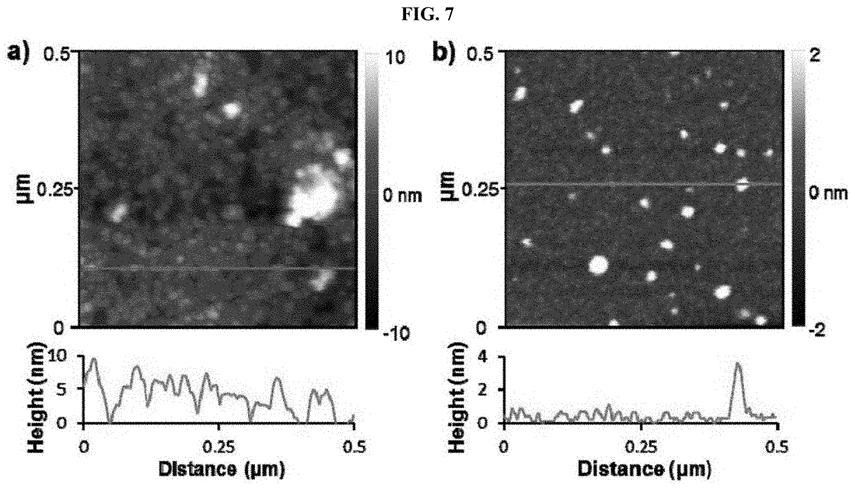

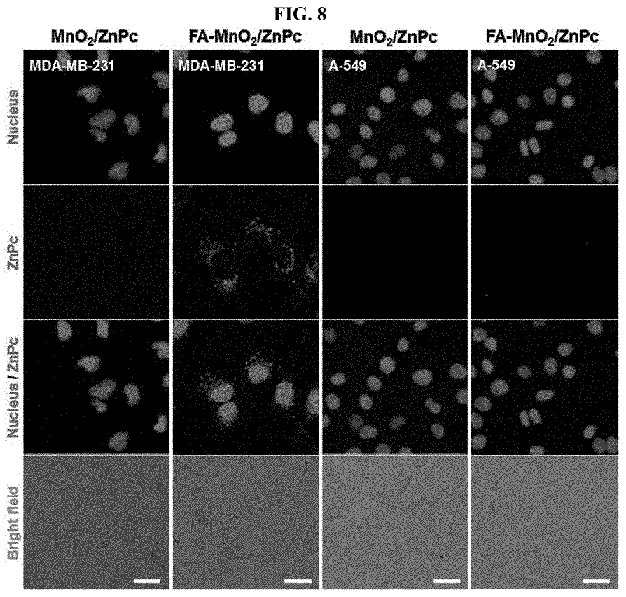

In this regard, ZnPc is loaded to FA-MnO₂ by electrostatic interaction and Mn—N coordination. It was demonstrated that the FA-MnO₂/ZnPc complex shows red displacement peaks in UV-Vis absorption spectra at 627 and 729 nm wavelengths, fluorescence extinction of ZnPc at 670 nm wavelength (λₑₓ=647 nm) , and a color change from dark yellow (FA-MnO₂) to light green (FA-MnO₂/ZnPc) in a properly dispersed solution. The optical characteristics described above mean that ZnPc was successfully loaded on FA-MnO₂. As a result of quantitative measurement, ZnPc was loaded in a capacity of at most 8 wt % on the FA-MnO₂ nanosheet (see FIG. 4A). AFM images showing the increased height without any coagulated structure have also supported that ZnPc was successfully loaded on the surface of the FA-MnO₂ nanosheet (FIG. 7).

[symbol]

### Experimental Example

### 1. Identification of Degradation of MnO2 by GSH and ZnPc Separation by the Same

Glutathione (GSH) as a reducing agent abundant in cytoplasm may induce degradation of MnO₂ to form Mn²⁺ cations. Therefore, in order to verify whether ZnPc can be easily separated from MnO₂ nanosheets entered into a cell, whether or not ZnPc is easily separated from the FA-MnO₂/ZnPc complex prepared in Example 3 has been determined by means of experiments.

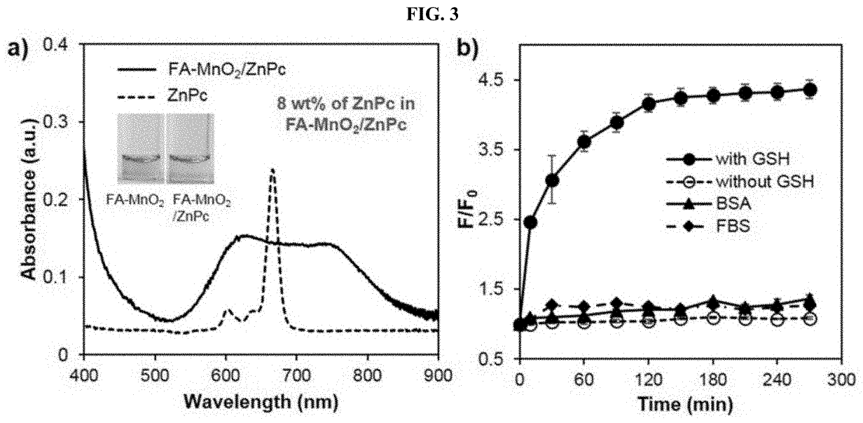

As a result, as shown in b) of FIG. 3, when adding

GSH to the FA-MnO₂/ZnPc solution, the eliminated fluorescence of ZnPc is gradually recovered, thus to demonstrate that degradation of the FA-MnO₂ mediated by GSH enables release of ZnPc from the FA-MnO₂/ZnPc complex. On the other hand, under non-GHS condition and in a case of adding a protein such as bovine serum albumin (BSA) or FBS, it could be found that degradation of the FA-MnO₂/ZnPc complex is almost not induced.

Accordingly, it is expected that the FA-MnO₂/ZnPc complex may be easily applied to physiological conditions by simple production of the complex even without binding any polymer or adding a surfactant to the complex.

[symbol]

### 2. Identification of Tumor Targeting by FA-MnO₂/ZnPc complex

In order to verify targeting abilities of the FA-MnO₂/ZnPc complex for FA-overexpressing cells, based on a fact that no significant change in characteristics of FA-MnO₂ in GSH (1 mM) at a low level corresponding to the blood level is observed, FR-α was overexpressed in HeLa cells, that is, human cervical cancer cells, followed by monitoring red fluorescence of ZnPc at 670 nm (λₑₓ=647 nm), so as to assess intracellular delivery of ZnPc by MnO₂ or FA-MnO₂. At this time, in order to block FA-mediated intercellular uptake of FA-MnO₂/ZnPc, before processing the FA-MnO₂/ZnPc, the surface of HeLa cells was pre-treated with free-FA for 2 hours in order to saturate FR.

As a result, as shown in FIG. 4A, strong red fluorescence of ZnPc appeared in the cytoplasm of the cells treated with FA-MnO₂/ZnPc, whereas cells treated with free-FA pre-treated MnO₂/ZnPc or FA-MnO₂/ZnPc did not show such significant fluorescence. These results indicate that ZnPc undergoes FA-mediated targeting by FA-MnO₂ and thus is delivered into the cells.

[symbol]

Next, in order to determine an extent of ZnPc absorption by the FA-MnO₂/ZnPc complex, quantitative measurement of the absorbed ZnPc was performed using a flow cytometer. FIG. 4B illustrates a cell population histogram in correlation with the fluorescence intensity of ZnPc in HeLa cells, while FIG. 4C shows calculation of a mean fluorescence intensity in each region and results thereof in a bar graph. Based on these results, FA-mediated ZnPc delivery by FA-MnO₂ could be quantitatively determined.

[symbol]

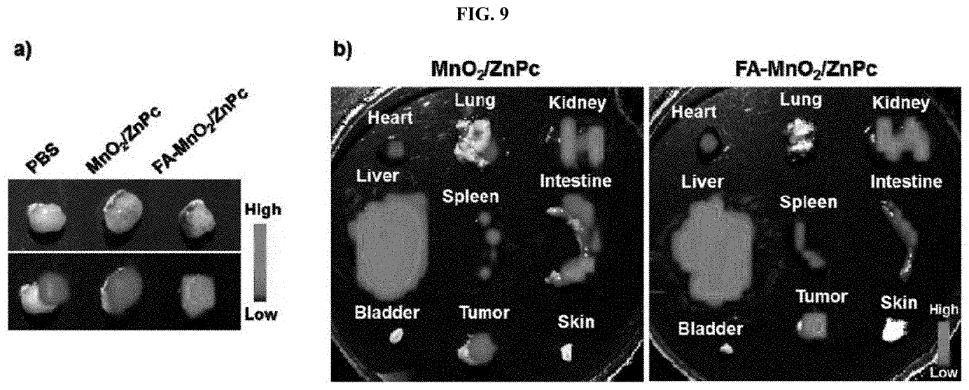

Furthermore, in order to identify that targeting by the FA-MnO₂ is based on interaction between FA and FR, FR-positive (FR+) and FR-negative (FR−) cells, that is, MDA-MB-231 cells (human breast cancer cell line) and A-549 cells (human alveolar basal epithelial cell line) were used, respectively, for observation of red fluorescence of ZnPc. As a result, strong red fluorescence of ZnPc was observed in the cytoplasm of FA-MnO₂/ZnPc-treated MDA-MB-231 cells, whereas MnO₂/ZnPc-treated MDA-MB-231 cells and MnO₂/ZnPc or FA-MnO₂/ZnPc-treated A-549 cells almost did not show fluorescence (FIG. 8). These results suggest that, through FA-FR interaction on the surface of FA-overexpressed cells, FA-MnO₂/ZnPc is effectively introduced into the cells by folate receptor-mediated endocytosis, whereby FA-MnO₂ can be used as an active target drug delivery system.

[symbol]

### 3. Identification of (In Vitro) Photodynamic Therapeutic Effects of FA-MnO2/ZnPc Complex

Based on the above experimental results, photodynamic therapeutic effects of the FA-MnO₂/ZnPc complex were investigated.

[symbol]

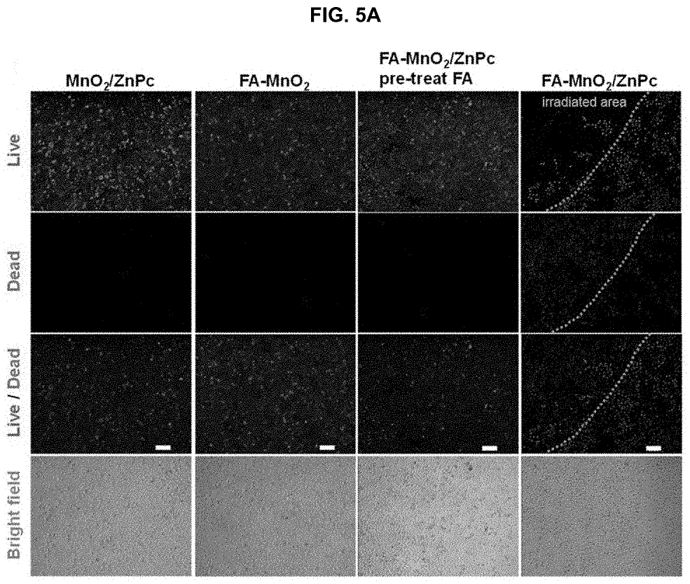

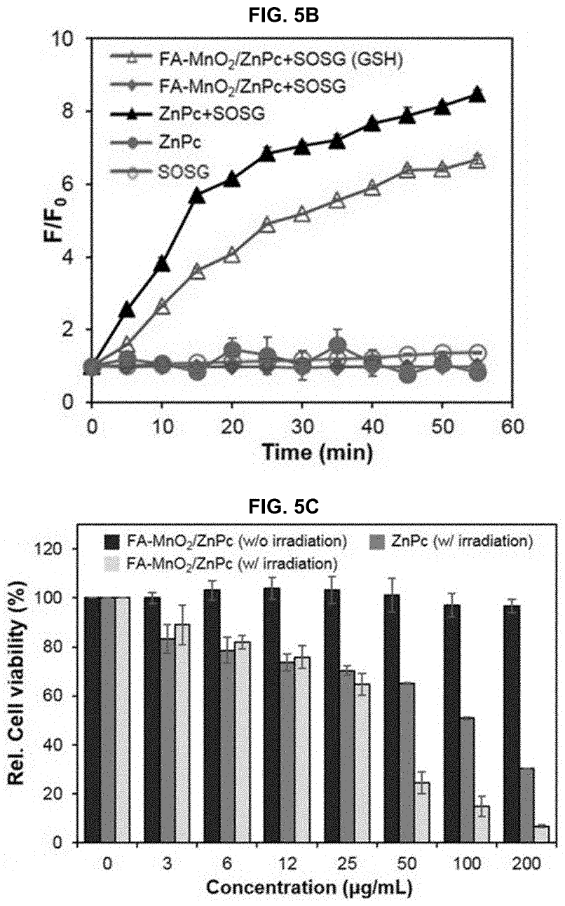

First, from the results of cell viability analysis in HeLa cells, as shown in FIG. 5A, in a case of FA-MnO₂/ZnPc treated cells, it was observed that red fluorescence at 572 nm (λₑₓ=547 nm) and green fluorescence at 520 nm (λₑₓ=490 nm), which are derived from dead cells and living cells, respectively, have a boundary clearly distinguished between irradiated region and non-irradiated region, unlike other cell groups showing green fluorescence only.

[symbol]

Next, under the background that green fluorescence of SOSG at 530 nm (λₑₓ=504 nm) is reinforced by SO (singlet oxygen) generation, Such SO generation was quantitatively investigated. Consequently, as shown in FIG. 5B, it was demonstrated that SO is generated by GSH-mediated MnO₂ degradation and photo-induced ZnPc activation, and this result is consistent with the results shown in b) of FIG. 3.

[symbol]

Finally, after conducting light irradiation into the entire well containing HeLa cells treated with FA-MnO₂/ZnPc at different concentrations, CCK-8 cell viability assay were subjected in order to assess dose-dependent PDT effects. As a result, as shown in FIG. 5C, it was demonstrated that IC₅₀ value of FA-MnO₂/ZnPc was 38.1±2.2 μg/ml, which is lower than IC₅₀ value of ZnPc alone, that is, 115.1±3.4 μg/ml.

These results suggest that FA-MnO₂/ZnPc has preferable PDT effects in living cells.

[symbol]

### 4. Identification of (In Vivo) Tumor Targeting and Anti-Cancer Effects of FA-MnO2/ZnPc Complex

Based on in vitro tumor targeting and photodynamic therapeutic effects of the FA-MnO₂/ZnPc complex, tumor tissue-targeted accumulation and in vivo anticancer effects were investigated using a mouse model.

[symbol]

First, in order to monitor in vivo distribution of ZnPc delivered by the FA-MnO₂ nanosheet, mice having tumor were prepared by the methods in Examples 1 to 9, and MnO₂/ZnPc, FA-MnO₂/ZnPc and PBS (control group) were intravenously injected. After 12 hours, observation for fluorescent signals in the tumor was conducted.

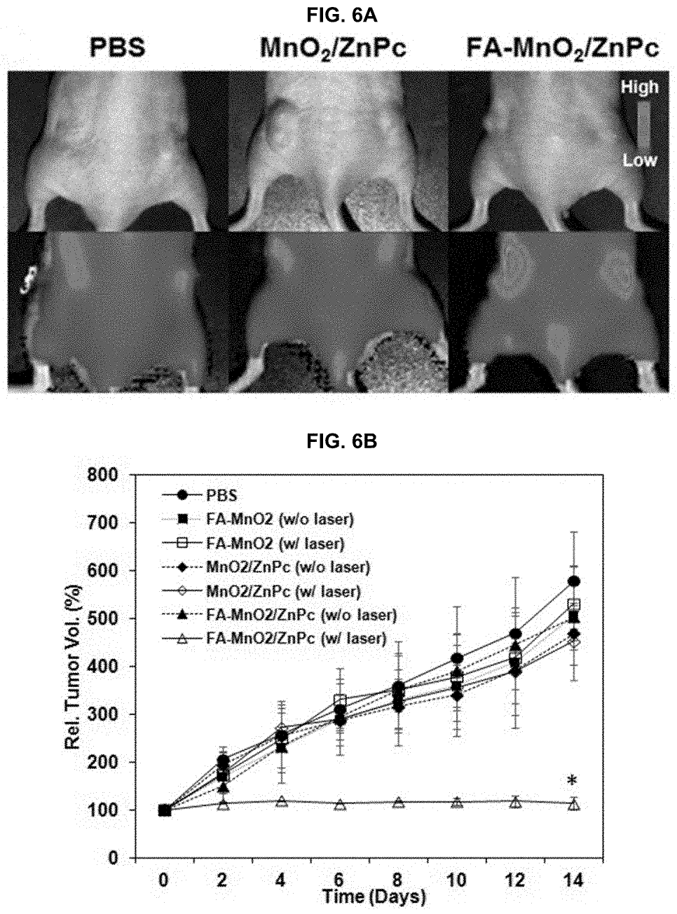

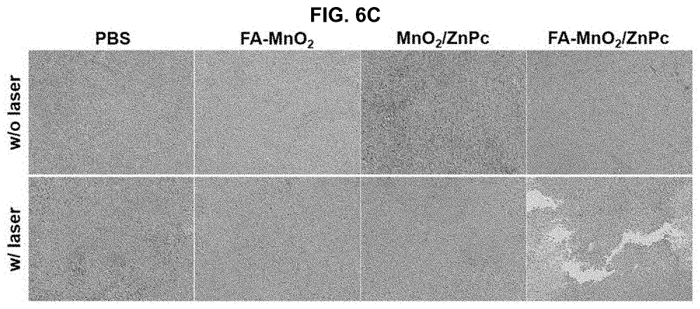

Consequently, as shown in FIG. 6A, strong red fluorescence of ZnPc was observed in the tumor treated with FA-MnO₂/ZnPc, whereas the tumor treated with MnO₂/ZnPc or PBS did not show such fluorescence.

[symbol]

Further, as compared to other tumor groups, FA-MnO₂/ZnPc-treated tumor tissue sections isolated from mice exhibited remarkable fluorescence signals (a) of FIG. 9). In addition, red fluorescence of ZnPc could be found in the liver of each of MnO₂/ZnPc and FA-MnO₂/ZnPc-treated mice (b) of FIG. 9). Furthermore, MnO₂/ZnPc nanosheet with a size of 150 nm was identified to be easily accumulated in the liver and spleen. These results suggest that FA-MnO₂ is more effective in targeted accumulation in FR-positive tumor, as compared to MnO₂ not modified in vivo.

[symbol]

Finally, photodynamic anticancer effects of FA-MnO₂/ZnPc in a human cancer xenograft model were investigated.

After growing the volume of each tumor up to 50 mm³, FA-MnO₂, MnO₂/ZnPc, FA-MnO₂/ZnPc (0.5 mg ZnPc/kg, 100 μl) and PBS were intravenously injected, respectively.

After 12 hours, light irradiation was conducted for 10 minutes by means of 660 nm laser (0.2 mW cm⁻²), followed by monitoring a change in tumor volume in each group for 2 weeks.

Consequently, as shown in FIG. 6B, the mouse treated with FA-MnO₂/ZnPc and subjected to laser irradiation has significant suppression in tumor growth. On the other hand, regardless of the laser irradiation, the groups treated with FA-MnO₂ or MnO₂/ZnPc did not show a significant difference in the tumor volume, as compared to the control group. These results suggest that FA-MnO₂/ZnPc-mediated PDT may efficiently induce apoptosis and also inhibit the growth of tumor.

[symbol]

On the basis of the above results showing statistically significant differences between the group treated with FA-MnO₂/ZnPc followed by laser irradiation and the remaining groups, tumor sections were subjected to H&E staining, 14 days after treatment using FA⁻MnO₂, MnO₂/ZnPc, FA-MnO₂/ZnPc and PBS, respectively.

Consequently, as shown in FIG. 6C, in contrast to the MnO₂/ZnPc, FA-MnO₂ or 1x PBS-treated group, remarkably increased apoptosis was observed in the tumor group treated with FA-MnO₂/ZnPc (laser irradiation) only.

[symbol]

Overall, it was verified that the FA-MnO₂-based PS delivery system of the present invention may enable efficient targeting to deliver ZnPc to a tumor site along with laser irradiation, so as to achieve high anticancer effects even by administration of only 10% of PS (0.5 mg kg⁻¹), compared to typically effective dose (5.0 mg kg⁻¹). Accordingly, FA-MnO₂-based target delivery of the present invention may achieve excellent biocompatibility, bio-imaging capability, targeting ability and therapeutic effects, and thus is expected to serve as an effective platform of improved PDT for treatment of FR-positive cancer.

[symbol]

[symbol]

1. A pharmaceutical composition for cancer treatment, comprising:
a metallic nanosheet to be degraded in a tumor tissue;
a folic acid to be bound to a folate receptor on a surface of a tumor cell; and
a photosensitizer excited by light irradiation at a predetermined wavelength,
wherein the folic acid and the photosensitizer are distributed on a surface of the metallic nanosheet.

2. The pharmaceutical composition according to claim 1, wherein the metallic nanosheet contains gold, silver, copper, platinum, palladium, nickel, iron, manganese or oxides thereof.

3. The pharmaceutical composition according to claim 1, wherein the metallic nanosheet contains manganese dioxide (MnO₂).

4. (canceled)

5. The pharmaceutical composition according to claim 1, wherein the photosensitizer is selected from the group consisting of phthalocyanine compounds, porphyrin compounds, chlorine compounds, bacteriochlorine compounds, naphthalocyanine compounds and 5-aminolevuline ester compounds.

6. The pharmaceutical composition according to claim 1, wherein the photosensitizer is zinc-phthalocyanine (ZnPc).

7. The pharmaceutical composition according to claim 1, wherein the predetermined wavelength ranges from 600 nm to 800 nm.

8. The pharmaceutical composition according to claim 1, wherein the excited photosensitizer generates singlet oxygen or free radicals.

9. The pharmaceutical composition according to claim 1, wherein the photosensitizer is contained in an amount of 5 to 20% by weight based on a total weight of a folic acid-metallic nanosheet-photosensitizer complex.

10. The pharmaceutical composition according to claim 1, wherein the metallic nanosheet contains manganese dioxide (MnO₂), and the photosensitizer is zinc-phthalocyanine (ZnPc).

11. The pharmaceutical composition according to claim 10, wherein the zinc-phthalocyanine is bound to the metallic nanosheet through a Mn—N coordinate bond.

12. The pharmaceutical composition according to claim 10, wherein the photosensitizer is bound to the metallic nanosheet by mixing particle size-reduced powders with a FA-MnO₂ solution and agitating the mixture.

13. A method for treating cancer, comprising administering a subject in need thereof the pharmaceutical composition according to claim 1, and irradiating light to excite the photosensitizer, wherein the cancer is any one or more selected from the group consisting of skin cancer, oral cancer, gastric cancer, ovarian cancer, breast cancer, osteosarcoma, colon cancer, esophageal cancer, duodenal cancer, renal cancer, lung cancer, pancreatic cancer, cervical cancer and prostate cancer.

14. A method for manufacturing a pharmaceutical composition for cancer treatment, comprising:
distributing folic acid on a surface of a metallic nanosheet; and
distributing a photosensitizer on the surface of the metallic nanosheet having the folic acid distributed thereon.

15. The method according to claim 14, wherein the metallic nanosheet contains MnO₂, and the photosensitizer is zinc-phthalocyanine.

16. The method according to claim 15, further comprising reducing a particle size of the photosensitizer powder before distribution of the photosensitizer.

17. A method for treating cancer, comprising:
administering a subject in need thereof the pharmaceutical composition according to claim 1; and
irradiating light at a predetermined wavelength to excite the photosensitizer.

18. The method of claim 17, wherein the metallic nanosheet contains gold, silver, copper, platinum, palladium, nickel, iron, manganese or oxides thereof.

19. The method of claim 17, wherein the photosensitizer is selected from the group consisting of phthalocyanine compounds, phorphyrin compounds, chlorine compounds, bacteriochlorine compounds, naphthalocyanine compounds and 5-aminolevuline ester compounds.

20. The method of claim 17, wherein the metallic nanosheet contains manganese dioxide (MnO₂), and the photosensitizer is zinc-phthalocyanine (ZnPc).

21. The method of claim 17, wherein the predetermined wavelength ranges from 600 nm to 800 nm.
